(19) United States
(12) Patent Application Publication
(10) Pub. No.: US 2008/0261959 A1
(43) Pub. Date: Oct. 23, 2008

(54) Novel crystalline forms of (S)-N-(1-Carboxy-2-methyl-prop-1-y)-N-pentanoyl-N[2'-(1H-tetrazol-5-yl)bi-phenyl-4-ylmethyl]-amine

(76) Inventors: Ashok Kumar, Mumbai (IN); Manmohan Madhavrao Nimbalkar, Mumbai (IN); Priti Jayesh Bhayani, Mumbai (IN); Mukesh Subhodh Jha, Mumbai (IN); Vaibhav Chinubhai Doshi, Mumbai (IN)

Correspondence Address: BLANK ROME LLP, 600 NEW HAMPSHIRE AVENUE, N.W., WASHINGTON, DC, 20037, US

(21) Appl. No.: 11/984,717

(22) Filed: Nov. 21, 2007

Related U.S. Application Data
(63) Continuation-in-part of application No. PCT/IN2006/000715, filed on May 25, 2005.

(30) Foreign Application Priority Data
May 25, 2005 (IN) 632/MUM/2005

(51) Int. Cl. A61K 31/41; C07D 257/04; A61K 31/549; A61K 31/4422; A61P 9/12

(52) U.S. Cl. 514/223.5; 548/253; 514/381; 514/356

(57) ABSTRACT

This invention relates to novel crystalline forms of valsartan, namely Form A, Form B, Form C, Form D and their solvates thereof. Processes for the preparation of the novel forms are also provided. The present invention further relates to novel processes for preparing a stable amorphous form of valsartan, and in this connection, to the amorphous form of valsartan produced by such processes. The present invention also discloses a novel process for obtaining stable Form I crystals of valsartan.

This application is a continuation-in-part (CIP) of PCT/IN06/00175, filed May 25, 2005, pending, which is incorporated herein by reference.

### FIELD OF INVENTION

This invention relates to novel crystalline forms of Valsartan namely, novel crystalline form of valsartan designated as Form A, and its solvates thereof, novel crystalline form of Valsartan designated as Form B and solvates thereof, novel crystalline form of Valsartan designated as Form C and solvates thereof, novel crystalline form of Valsartan designated as Form D and its solvates thereof, processes for their preparation, pharmaceutical compositions containing these polymorphs and their use in medicine. The present invention further relates to a novel processes for preparing a stable amorphous form of Valsartan and in this connection to amorphous form of Valsartan produced by such processes. The present invention also discloses a novel process for obtaining stable Form I crystals of Valsartan.

### BACKGROUND OF THE INVENTION

(S)-N-(1-Carboxy-2-methyl-prop-1-yl)-N-pentanoyl-N-[2′-(1H-tetrazol-5-yl)bi-phenyl-4-ylmethyl]-amine commonly known as Valsartan has the following structure (Formula I):

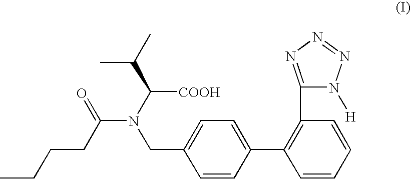

Valsartan is a member of the class of agents termed angiotensin-II (AT) receptor antagonists having effective anti-hypertensive activity with an excellent profile of safety and tolerability. Activation of AT receptors in the outer membrane of vascular smooth muscle cells of the heart and arteries causes the tissues to constrict. AT-I receptors are activated by an octa-peptide, angiotensin-II. Angiotensin-II helps to maintain constant blood pressure despite fluctuations in a person's state of hydration, sodium intake and other physiological variables. Angiotensin-II also performs the regulatory tasks of inhibiting excretion of sodium by the kidneys, inhibiting nor-ephedrin reuptake and stimulating aldosterone biosynthesis. By inhibiting angiotensin-II binding to AT receptors, valsartan disrupts the vasoconstriction mediated by AT receptors.

Valsartan is therefore a non-peptide angiotensin-II antagonist, inhibits the actions of angiotensin-II on its receptors, thus preventing the increase of blood pressure produced by the hormone-receptor interactions. Hence it is used in the treatment of cardiovascular complaints such as hypertension and heart failure. Comparative trial studies have shown that valsartan is as effective as angiotensin-converting enzyme (ACE) inhibitors, calcium-channel blockers and beta-blockers, and is generally better tolerated. Valsartan is marketed as the free acid under the name trade name DIOVAN, however, its combination with diuretics, such as hydrochlorothiazide have specific advantage as anti-hypertensive agent.

The synthesis of Valsartan and its intermediates was reported in patent (U.S. Pat. No. 5,399,578) and Bioorganic & Medicinal Chemistry Letters, vol. 4, pp 29-34, 1994. However this patent fails to disclose any crystalline forms of Valsartan.

The present invention relates to the solid state physical properties of Valsartan. The solid state properties can be changed by controlling the conditions under which Valsartan is obtained in solid form. Solid state physical properties influence, for example, the flowability/fluidity of the milled solid. Flowability affects the ease with which the material is handled during processing into a pharmaceutical product/composition. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate in the preparation.

Another important solid state property of a pharmaceutical compound is its rate of dissolution in aqueous/lipid fluid. The rate of dissolution of an active ingredient in a patient's stomach fluid can have therapeutic consequences when drugs administered orally since it imposes an upper limit on the rate at which an orally-administered active ingredient can reach the patient's bloodstream. The rate of dissolution as well as the equilibrium solubility are also an important consideration while formulating syrups, elixirs and other liquid medicaments since a polymorph may have little solubility in the medium and polymorphic changes can occur in presence of solvents. The solid state properties of a compound may also affect its behavior on compaction and its storage stability. The physical characteristics of a compound are influenced by the conformation and orientation of molecules in the unit cell, which defines a particular polymorphic form of a substance. The polymorphic form may give rise to different thermal behavior from that of the amorphous material or another polymorphic form.

Thermal properties of a pharmaceutical compound is measured in the laboratory by using techniques such as capillary melting point, thermogravimetric analysis (TGA) and differential scanning calorimetric (DSC) and can be used as a basic tool to distinguish some polymorphic forms from others. A particular polymorphic form will give rise to distinct spectroscopic properties that may be detectable by powder X-ray crystallography, solid state ¹³C NMR spectroscopy and infrared spectrophotometry.

A crystalline form of a substance has well-defined crystal lattices and distinct spectral characteristics when subjected to X-Ray crystallography; however, an amorphous form will exhibit a “smearing” of some of those properties due to the lack a specific crystal order. An amorphous substance will produce a near featureless PXRD pattern although the diffraction pattern of a crystalline form of the same substance may have many well-resolved reflections. Generally, molecular interactions caused by tight crystal packing make a substance more thermally stable and less soluble than the substance in an amorphous state. Although thermal stability is a desirable characteristic of a pharmaceutical compound, it is often the case that increased, rather than decreased, solubility is desired. But a decreased solubility instead of rapid dissolution may be desired in formulating dosage regimen for delayed release of a particular medicament. The rate of dissolution of an active ingredient in a patient's gastric fluid can have therapeutic consequences since it imposes an upper limit on the rate at which an orally-administered active ingredient can reach the patient's bloodstream. Increased solubility of a pharmaceutical agent in aqueous fluids, therefore, can increase bioavailability. The effect that the solid-state has on bioavailability may be so significant that a crystalline form of a drug cannot be considered bioequivalent to the amorphous form.

In U.S. Pat. No. 5,399,578 example 16, the Valsartan obtained from ethyl acetate indicated to have melting intervals ranging from 105 to 115° C. In the Merck index, Valsartan is described as crystals from di-isopropyl ether having a melting point of 116 to 117° C. However, we have obtained valsartan from diisopropyl ether as a sticky solid which upon drying yielded Valsartan having a melting range of 70 to 79° C. and was characterized to be amorphous by PXRD analysis.

It is further substantiated by findings of Marti et al (Please refer WO 02/06253, page 2, paragraph 1) that the X-ray diffraction pattern of valsartan free acid obtained from prior process consists essentially of a very broad, diffused X-ray reflection; and therefore designated as almost amorphous under X-Ray powder diffraction.

Subsequently WO03/089417 disclosed new crystalline forms of Valsartan designated as ‘Form I’ and ‘Form II’ and their preparation methods.

Yet another patent application WO04/083192 disclosed new crystalline forms of Valsartan designated as ‘Form I to Form 1× and methods for their preparation. This patent also discloses pure amorphous form of valsartan having a differential scanning calorimetry (DSC) thermogram lacking melting endotherm above 1 J in the range of 80 to 100 degree Celsius, and a process for the preparation of the same. The patent also discloses about inter-conversion of one crystalline form to another crystalline form or vice versa. It has been observed that the crystal forms claimed in both WO03/089417 and WO04/083192 are found to be contaminated with high contents of amorphous valsartan.

Thus it is clear from the foregoing discussion, it would be desirable to have valsartan in a stable crystalline form having improved bulk handling and dissolution properties and this becomes the object of the present invention

### SUMMARY OF THE INVENTION

It has now been found that Valsartan can exist, in addition to known crystalline forms, in other crystalline forms stable at room temperature, in particular, in the novel crystalline forms with higher percentage crysallinity described herein after.

In one aspect, the present invention relates to a new crystalline form of Valsartan which is hereinafter designated as Form A and its solvates thereof. More particularly, the present invention relates to a novel crystalline form of Valsartan denoted as Form A as characterized by a powder X-ray diffraction pattern with peaks at about 6.7488, 14.237, 20.87, 21.807 and 22.256 degrees 2 theta angles. The ‘Form A’ is also characterized by a DSC thermogram having a melting interval at 95 to 96° C. temperature. The Valsartan ‘Form A’ of the present invention has a crystal content of at least 20%, preferably 40% and more preferably 50% or more having characteristic peak at 6.7488 degrees 2theta angle in a PXRD diagram and a DSC thermogram having an endotherm at 95 to 96° C.

In a further aspect, the present invention relates to a process for preparing Valsartan in crystalline ‘Form A’ including the steps of providing a solution of Valsartan in a first solvent selected from acetone, methyl propyl ketone or their mixture thereof, bringing the valsartan solution to a temperature of about 25 to 35° C., adding a second solvent, preferably an antisolvent, such as dichloromethane, whereby a suspension is formed and cooling the suspension/solution to isolate Valsartan in ‘Form A’ by filtration.

In another aspect, the present invention also relates to one more new crystalline form of Valsartan, which is characterized by a powder X-ray diffraction pattern, with a characteristic peak at about 5.810 degrees 2θ. The said new crystalline form is denoted as ‘Form B’. The new form of the present invention may also exist as solvates. More particularly the ‘Form B’ polymorph of Valsartan is characterized by a powder X-ray diffraction pattern with peaks at about 5.810, 9.815, 11.463, 13.937 and 17.420 degrees 2 theta values. Another characterization of this novel form is a differential scanning calorimetric thermogram having an endotherm at about 103° C.

In yet another aspect, the present invention further relates to a process for preparing Valsartan having at least one characteristic of ‘Form B, including the steps of providing an emulsion or suspension of valsartan in an organic solvent, such as toluene, at a first temperature, reducing the temperature of the emulsion or suspension to a second temperature, stirring the mixture at the second temperature for longer duration in the range of about 24 to 40 hours, further reducing the temperature of the stirred suspension to a third temperature range and isolating crystalline ‘Form B’ of Valsartan by filtration and drying.

In yet another aspect, the present invention relates to one more new crystalline form of Valsartan which is hereinafter designated as Form C and its solvates thereof. More particularly, the present invention relates to a novel crystalline form of Valsartan denoted as Form C as characterized by a powder X-ray diffraction pattern with peaks at about 13.85, 5.256, 7.443, 20.316, 24.017, 25.11, 12.800, 11.733, 9.662, 15.684, and 17.023±0.30 degrees 2 theta angles. The ‘Form C’ valsartan of the present invention is also characterized by a DSC thermogram having a melting interval having maxima between 106 to 113° C. The Valsartan ‘Form C’ of the present invention has a crystal content of at least 20%, preferably 50% and more preferably over 70% having characteristic peak at 7.443, 13.851, 12.800, 11.733, 14.683, 24.01 and 25.11±0.30 degrees 2 theta angle on a PXRD diagram and a DSC thermogram having an endotherm at 106 to 113° C.

In the above aspect, the present invention also relates to a process for preparing Valsartan in crystalline ‘Form C’ which includes the steps of suspending amorphous or partially crystalline valsartan in a hydrocarbon solvent, preferably toluene, at a temperature, preferably from 0-60 degree C., more preferably from room temperature to 60 degree where valsartan is stable to racemization; agitating the suspension for a period of 24 hours to 110 hours; filtering the crystals in the suspension to isolate Valsartan ‘Form C’ of the present invention.

In one more aspect, the present invention relates to a new thermodynamically stable crystalline form of Valsartan which is hereinafter designated as Form D and its solvates thereof. More particularly, the present invention relates to another novel crystalline form of Valsartan denoted as Form D as characterized by a powder X-ray diffraction pattern with peaks at about 6.50, 11.58, 16.63, 19.53, 21.99 and 24.04±0.25 degrees 2 theta angles. The ‘Form D’ valsartan of the present invention is also characterized by a DSC thermogram having a melting interval having maxima between 129 to 135° C. The Valsartan ‘Form D’ of the present invention has a crystal content of at least 20%, preferably 50% and more preferably over 85% having characteristic peak at 6.50, 11.58, 16.63, 19.53, 21.99 and 24.04±0.25 degrees 2 theta angle on a PXRD diagram and a DSC thermogram having an endotherm at 129 to 135° C. The high melting valsartan ‘Form D’ was further characterized by FTIR absorptions at 1705, 1485, 1425, 1294, 824, 536, 678, and 666 which are absent in other forms.

In a further aspect, the present invention relates to a process for preparing Valsartan in crystalline ‘Form D’ which includes the steps of suspending amorphous or partially crystalline valsartan in a hydrocarbon solvent, preferably toluene, at a temperature, preferably 0 to 60 degree C., more preferably room temperature to 60 degree C., where valsartan is stable to racemization; agitating the suspension for a period of 115 hours or more, (which may extend to several days); filtering the crystals from the suspension to isolate stable valsartan ‘Form D’ of the present invention.

Alternately, ‘Form D’ crystals can be obtained by suspending amorphous or partially crystalline valsartan in toluene or its combination with other solvents, such as water, ethyl acetate, or xylene, in the presence of seeds of ‘Form D’ crystals of Valsartan; agitating the mixture for a period of 15 to 28 hours and isolating ‘Form D’ crystals of valsartan.

Furthermore, the present invention provides a process for preparation of amorphous form of Valsartan characterized by a differential scanning calorimetry thermogram having no endotherms.

In yet another aspect, the present invention relates to a process for preparing crystalline Valsartan denoted as ‘Form I’ including the steps of dissolving Valsartan in methyl propyl ketone solvent at about 50 to 55° C. to form a solution, cooling the solution to about 25 to 35° C. and further cooling to about 0 to 5° C. to obtain crystals of Valsartan in ‘Form I’ as disclosed in WO 03/089417, which is incorporated herein by reference.

In another aspect, the present invention relates to pharmaceutical compositions containing such novel crystalline Valsartan ‘Form A’ or ‘Form B’ or ‘Form C’ or ‘Form D’ or amorphous form and their solvates thereof for producing an anti-hypertensive/cardiovascular effect in mammals, including human patients for treating hypertension. Valsartan ‘Form A’ or ‘Form B’ or ‘Form C’ or ‘Form D’ or amorphous form, and their solvates thereof can be formulated into a variety of compositions for administration to humans and mammals. Pharmaceutical compositions of the present invention contain Valsartan ‘Form A’ or ‘Form B’ or ‘Form C’ or ‘Form D’ or amorphous form and their solvates thereof, optionally as mixtures with other crystalline forms and/or other active pharmaceutical drugs such as diuretic like hydrochlorothiazide or calcium channel blockers like amlodipine or their pharmaceutically acceptable salts and such synergic compositions resulting from such compositions. In addition to the active pharmaceutical ingredient (s), the pharmaceutical compositions of the present invention can contain one or more commonly used pharmaceutical excipients. These excipients are added in the composition for a variety of purposes.

### BRIEF DESCRIPTION OF THE ACCOMPANYING DRAWINGS

FIG. 1 represents Powder X-Ray diffraction pattern (PXRD) of ‘Form A’ of Valsartan.

FIG. 2 represents Powder X-Ray diffraction pattern (PXRD) of ‘Form B’ of Valsartan.

FIG. 3 represents Powder X-Ray diffraction pattern (PXRD) of ‘Form C’ of Valsartan.

FIG. 4 represents Powder X-Ray diffraction pattern (PXRD) of ‘Form D’ of Valsartan.

FIG. 5 represents Powder X-Ray diffraction pattern (PXRD) of ‘Form I’ of Valsartan prepared according to example 3 of the present invention.

FIG. 6 represents Powder X-Ray diffraction pattern (PXRD) of ‘amorphous’ form of Valsartan prepared according to example 4 of the present invention.

FIG. 7 represents Differential Scanning Calorimeter thermogram of ‘Form A’ of Valsartan.

FIG. 8 represents Differential Scanning Calorimeter thermogram of ‘Form B’ of Valsartan.

FIG. 9 represents Differential Scanning Calorimeter thermogram of ‘Form C’ of Valsartan.

FIG. 10 represents Differential Scanning Calorimeter record of ‘Form D’ of Valsartan.

FIG. 11 represents Differential Scanning Calorimeter record of ‘amorphous’ form of Valsartan prepared according to example 4 of the present invention

FIG. 12 represents an overlay of PXRD pattern of Form C having varying crystal content after conversion of amorphous material at 24, 48, 72 and 96 hours in toluene.

FIG. 13 shows an overlay of PXRD patterns of polymorph ‘Form C’ and ‘Form D’ of Valsartan

FIG. 14. Represents an overlay of the Infra red absorption spectra of ‘Form D’ Valsartan and Valsartan obtained as per U.S. Pat. No. 5,399,578.

FIG. 15 represents a comparative plot of equilibrium solubility of crystalline ‘Form D’ valsartan and amorphous valsartan at intervals of 1-0, 15, 30, 45, and 60 minutes.

FIG. 16, represents a plot of the comparative intrinsic dissolution profile of crystalline ‘Form D’ valsartan and amorphous valsartan in 1.2, 4.5, and 6.8 pH buffer media.

### DETAILED DESCRIPTION OF THE INVENTION

As used herein, the phrase “Valsartan ‘Form A or Form B’ or ‘Form C’ or ‘Form D’” where ‘A’, ‘B’, ‘C’, and ‘D’ are letters refers to a crystalline forms of Valsartan that one of skill in the art can identify as a distinct entity distinguishable from other crystalline forms of Valsartan based on the characterization provided herein. As used herein, the phrase having “at least one characteristic of Form A’, or ‘Form B’ or ‘Form C’ or ‘Form D’ refers to a crystalline form of Valsartan that possesses one of the PXRD peaks or endotherms of a DSC thermogram provided herein. For example, a single or a combination of PXRD peaks which is not found in another crystalline form of Valsartan is enough to show at least one of the characteristics of Form ‘A’ or ‘B’ or ‘C’ or ‘D’. A single or a combination of endotherms of a DSC thermogram may also serve the same purpose.

The DSC, FTIR and PXRD methods used for the identification and characterization of the new polymoprhs of Valsartan are described below:

### a) Differential Scanning Calorimetry

DSCs were recorded using a TA Q100 instrument with a standard open aluminium pan, calibrated using indium and zinc standards. Samples (2.0 mg) were accurately weighed into DSC pans; the DSC profiles were recorded at different heating rate (2, 5, 10° C./min), range from 20 to 200° C., under nitrogen flux. The weight of samples was about 2 mg and the samples were scanned at a heating rate of 5° C./min from 0 to 160° C. under a nitrogen flush. The DSC experiments were run using pans that were open, closed, or closed with a corner hole.

### b) FT-IR Spectral Analysis

FTIR spectra of all the crystal forms were obtained using a dispersion (0.5%) in alkali Halide (KBr) disk and directly on untreated powder by means of spectrometer. Spectra was recorded at room temperature from 4000 cm⁻¹ to 650 cm⁻¹, for each sample 32 scans were collected at a resolution of 4 cm⁻¹.

### c) X-Ray Powder Diffraction Studies.

The PXRD pattern was measured on a SIEMAN D500 40 KV/30 mA powder X-Ray Diffractometer, with a solid state detector, in the 2 theta angles range between 3 to 400. Copper (Cu Kα) radiation-Ni filtered of 1.5406 AO wavelength was used. The step scan mode was performed with a step width of 0.02°, at a scan rate of 0.5 step/s.

The term ‘stable’ as used herein, refers to the tendency to remain substantially in the same physical form for at least a month, preferably at least 6 months, more preferably at least a year, still more preferably at least 3 years, even still more preferably at least 5 years, when stored under ambient conditions (25[deg.] C./60% RH) without external treatment. It should be noted that the amorphous forms of many compounds sometimes revert to the partly crystalline form in a relatively short time period (days/weeks rather than months/years), and therefore not stable in many cases under normal storage conditions implicating the significance of the present invention. Substantially the same physical form in this context means that at least 70%, preferably at least 80% and more preferably at least 90% of the crystalline form remains.

In one embodiment, the crystalline form of valsartan of the present invention is substantially free from amorphous forms of valsartan or other forms. “Substantially free,” from other forms or amorphous form of valsartan, shall be understood to mean that crystalline valsartan contains less than 50%, preferably less than 25%, more preferably less than 10% and still more preferably less than 5% of other forms of valsartan, e.g. amorphous valsartan.

It has been seen that amorphous valsartan tends to form a glassy/sticky solid and has very poor dispersion properties especially when contacted in aqueous or polar solvents. Now it has surprisingly been found that the substance valsartan can be prepared in a stable crystalline form. Moreover, it has been found that crystalline valsartan possesses far greater handling properties and stability than the amorphous form. Furthermore the solubility profile of new crystalline forms are much better than the rapid dissolution or wetting properties of amorphous valsartan. Such a controlled solubility or handling properties of crystalline form of valsartan may render the product not only more suitable to certain formulations where sustained solubility is desired, such as ‘slow release or once-a-day’ formulations, but also suitable for conventional formulations.

Although partly crystalline and amorphous form of valsartan has been in the public domain for some time now, the applicants are not aware of any disclosure of more pure crystalline valsartan having been made and publicly disclosed. Indeed, it was generally regarded that crystalline valsartan would be difficult to make, particularly in a stable crystalline form (please see WO02/06253 and U.S. Pat. No. 6,294,197, which are incorporated herein by reference). Applicants' previous attempts at crystallization to produce other forms, always generated partly crystalline or amorphous form that did not alter the undesired properties of amorphous form to a desirable extent. No pure crystalline product was ever formed.

The crystalline forms of the present invention have easily dispersible granular particles and better bulk density as opposed to the amorphous valsartan particles which are glassy in appearance having disordered surfaces with the absence of regular faces present in crystalline materials. Generally granular nature of the crystalline particles will impart improved flow characteristics and so aid tablet manufacture compared to the glassy disordered structures found in the amorphous material. Sometimes tablet manufacture by direct compression, as opposed to wet granulation, is prone to segregation of the drug substance from the remaining, excipients, leading to a non-uniform mix. This gives rise to tablets of variable drug content. Segregation is exacerbated by wide differences in the particle size of the drug substance and the excipients. The larger particle size of the crystalline valsartan compared to the amorphous material would be closer to that of the excipients typically used in direct compression formulations and so would minimize segregation. Further, it is well known that crystalline materials posses improved compression and formulation characteristics over the amorphous form in oral solid dosage forms.

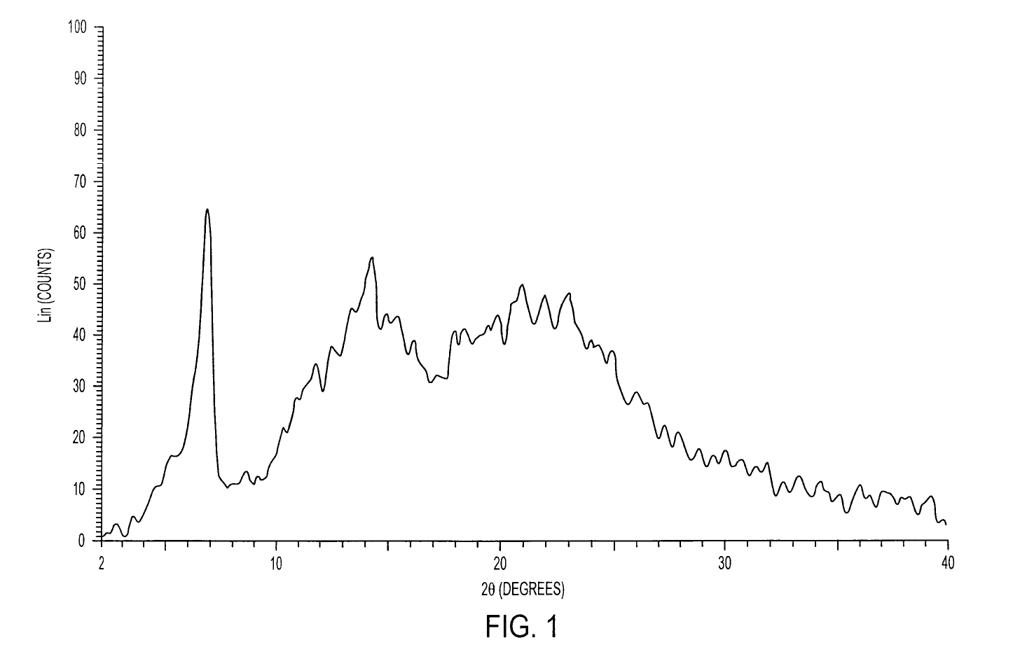

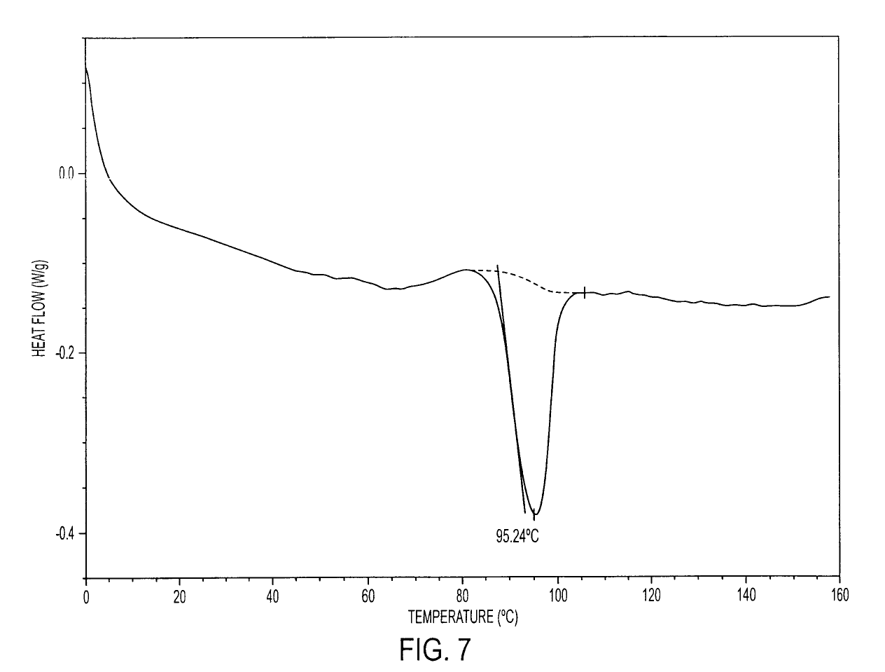

Thus, according to the major objective of the invention there is provided valsartan in crystalline form. In one aspect, this invention provides novel Valsartan in a specific and distinguishable crystalline form that is denoted as “Valsartan Form A”. The character of this new form is confirmed by PXRD patterns and Differential Scanning Calorimeter (also referred as DSC) obtained from a sample thereof which are provided as FIGS. 1 and 7 respectively. The PXRD pattern shows at least one characteristic peak at about 6.7488 degrees 2θ. More particularly the PXRD pattern shows characteristic peaks at 6.7488, 14.237, 20.87, 21.807 and 22.256 degrees 2θ. Further, DSC shows a characteristic endotherm at about 95 to 96° C. for Form A. DSC was measured in a TA Q100 instrument using a standard open pan. The weight of samples was about 2 mg and the samples were scanned at a heating rate of 5° C./min from 0 to 160° C. under a nitrogen atmosphere.

The crystalline ‘Form A’ of Valsartan typically has X-ray powder diffraction pattern as substantially as shown in the FIG. 1 and the characteristic peaks with their 2θ value and corresponding d spacing are listed in Table 1 below.

TABLE 1

| 2θ values in degrees | d spacing |
|---|---|
| 6.7488 | 13.08752 |
| 14.237 | 6.22 |
| 20.87 | 4.25 |
| 21.807 | 4.072 |
| 22.256 | 3.887 |

In one embodiment, the present invention provides a process for preparing Valsartan in a crystalline form that denoted as ‘Form A’ having at least one characteristic listed in table 1. The process includes the steps of providing a solution of Valsartan in a first solvent selected from acetone, methyl propyl ketone or their mixture thereof, mixing a second solvent, preferably an antisolvent, such as dichloromethane, to the valsartan solution whereby a suspension is formed and cooling the suspension/solution to obtain valsartan in ‘Form A’. The process further includes the steps of reducing the temperature of the Valsartan solution and maintaining the suspension at reduced temperature for a holding time, preferably 30 minutes to 3 hours, more preferably 30-60 min.

In a preferred embodiment of the present invention, Valsartan is dissolved in a solvent, such as acetone or methylpropyl ketone or their mixture thereof, at a temperature of about 30° C. to reflux temperature to form a solution in said solvent, followed by reducing the temperature of the solution to a temperature of about 25 to 35° C., and mixing with a second solvent, which may also be an antisolvent, such as dichloromethane, at 25 to 35° C. to form a suspension. The suspension obtained in the mixture of solvents may be further cooled to a temperature of −5 to +5° C. to obtain pure ‘Form A’ of Valsartan. The ‘Form A’ crystals can then be separated from the mixture by conventional means, such as filtration and can be optionally dried at ambient or elevated temperatures.

The Valsartan starting material can be dissolved in the solvent wherein heat may be used to effect dissolution. Preferably the starting material is dissolved at 30° C. to reflux temperature of the solvent. The most preferable temperature used for the dissolution of valsartan in acetone or methylpropyl ketone is about 40 to 45° C. and the second solvent addition is preferably carried out at a temperature of about 30 to 33° C.

The dissolution solvent is preferably used in about 2 to 3 volumes (mL) relative to the weight (g) of Valsartan and the second solvent e.g., dichloromethane is preferably used in about 5 to 10 volumes relative to the first dissolution solvent.

In an alternate embodiment of the present invention the amorphous, or partially crystalline or any crystalline form of valsartan can be converted to crystalline ‘Form A’ by way of the present process. More preferably an amorphous form of Valsartan is converted to a stable crystalline form (Form A) by using the process of the present invention.

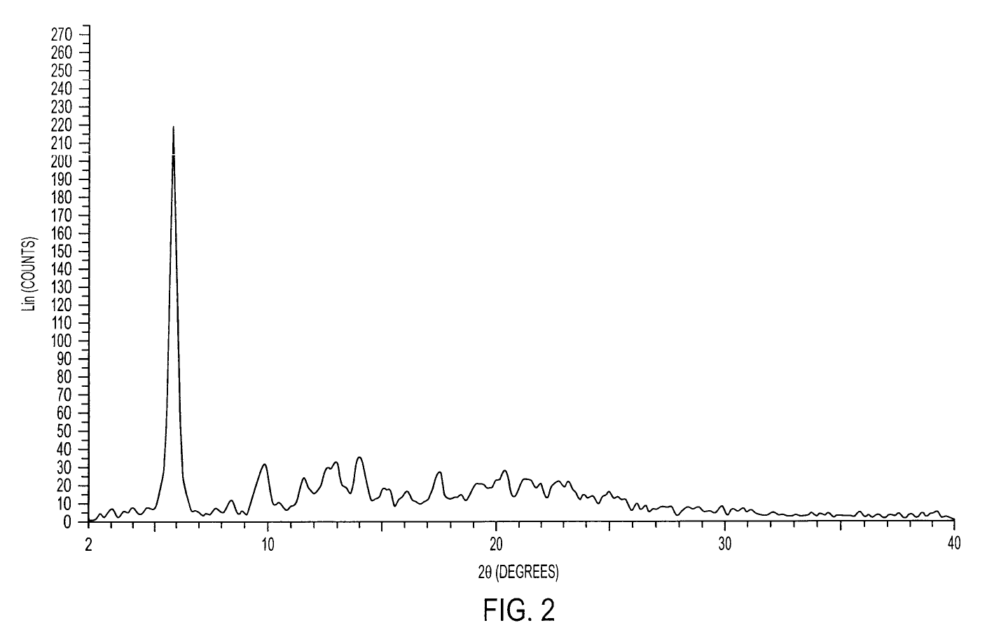

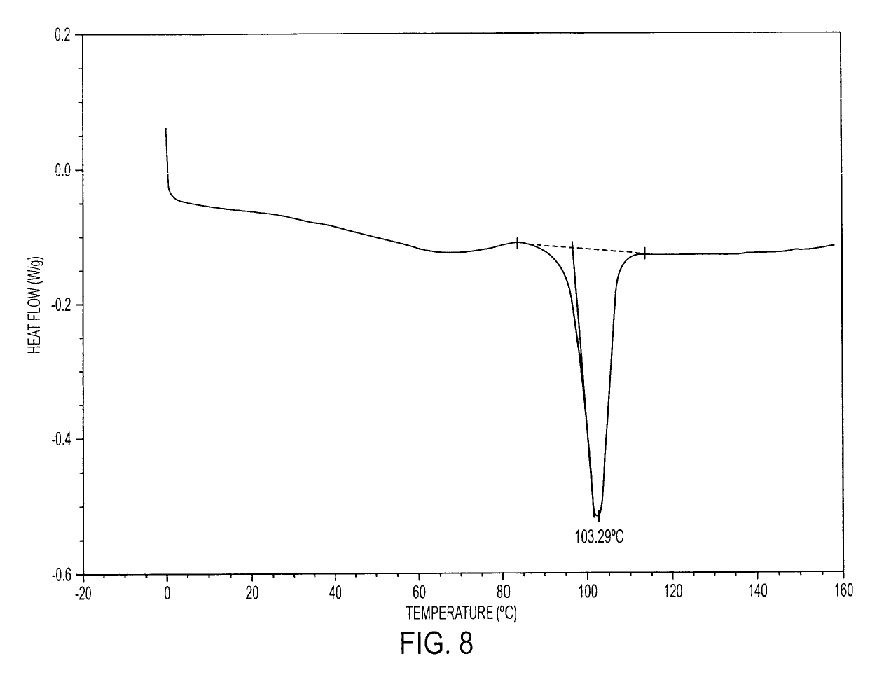

In a second aspect, this invention provides novel valsartan in a specific & distinguishable crystalline form that is denoted as “Valsartan Form B”. The character of this new form is confirmed by PXRD patterns and DSC obtained from a sample thereof which are provided in FIGS. 2 and 8 respectively. The PXRD pattern shows at least one characteristic peak at about 5.810 degrees 2θ. More particularly the PXRD pattern has characteristic peaks at 5.810, 9.815, 11.463, 13.937 and 17.420 degrees 2θ. Further, DSC thermogram of ‘Form B’ shows a characteristic endothermic peak at about 103° C. DSC was measured in a TA Q100 instrument using a standard open pan. The weight of samples was about 2 mg and the samples were scanned at a heating rate of 5° C./min from 0 to 160° C. under a nitrogen atmosphere.

The crystalline ‘Form B’ of Valsartan typically has X-ray powder diffraction pattern as substantially as shown in the FIG. 2 and the characteristic peaks with their 2θ values and corresponding d spacings are listed in Table 2 given below.

TABLE 2

| 2-theta values in degrees | d spacing values |
|---|---|
| 5.810, | 15.198 |
| 9.815, | 9.004 |
| 11.463, | 7.713 |
| 13.937 | 6.348 |
| 17.420 | 5.086 |

In another embodiment, the present invention provides a process for preparing valsartan and its solvates in a crystalline form that denoted as ‘Form B’ having at least one characteristic listed in Table 2. The present process includes the steps of providing an emulsion or suspension of Valsartan in an organic solvent like toluene at a first temperature, reducing the temperature of the emulsion or suspension to a second temperature, stirring the mixture at the second temperature for longer duration preferably in range of about 24 to 40 hours, followed by further reducing the temperature of the stirred suspension to a third temperature range to obtain crystalline ‘Form B’ of Valsartan and can be separated from the mixture by conventional means such as filtration and can be optionally dried at ambient or elevated temperatures.

The first temperature is preferably being about reflux temperature of the solvent and the second temperature, is more preferably in the range of about 25 to 35° C. The third temperature range characterized by isolation of ‘Form B’ of Valsartan is about −5 to +10° C.

In a preferred embodiment of the present invention, valsartan is emulsified in the solvents like toluene at reflux temperature to form an emulsion in said solvent followed by reducing the temperature of the solution to about 25 to 35° C. The mixture is maintained under constant stirring at 25 to 35° C. to form a suspension in duration of about 24 to 40 hours. The suspension of Valsartan obtained in the solvent toluene is further cooled to a temperature of −10 to 10° C. and further maintained for about 2 to 4 hours. The obtained crystals after filtration and drying yield pure ‘Form B’ of Valsartan. The emulsifying solvent is preferably used in about 8 to 12 volumes (mL) relative to the weight (g) of valsartan.

In an alternate embodiment of the present invention, the amorphous, or partially crystalline or any crystalline form of valsartan can be converted to crystalline ‘Form B’ by way of the present process. More preferably, an amorphous form of Valsartan is converted to a stable crystalline form (Form B) by using the process of the present invention.

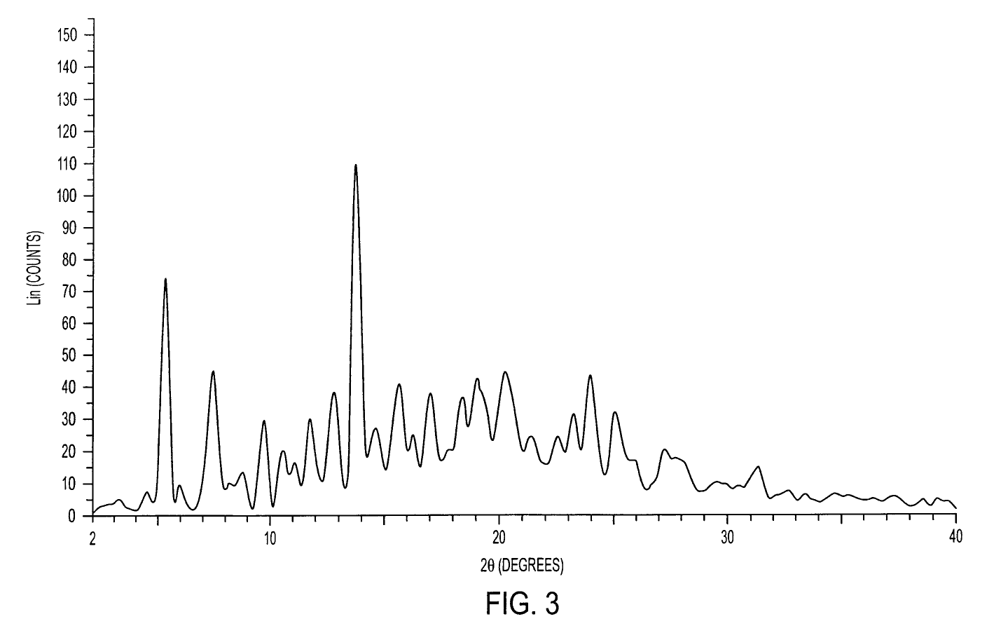

In one more aspect, this invention provides novel Valsartan in a stable, specific and distinguishable crystalline form that is denoted as “Valsartan Form C”. The character of this new form is identified and confirmed by PXRD patterns and Differential Scanning Calorimeter thermogram (also referred as DSC) obtained from a sample thereof which are provided in FIGS. 3 & 9, respectively. The PXRD pattern shows at least one characteristic peak at about 13.851±0.2 degrees 2θ. More preferably, the PXRD pattern shows characteristic peaks at 13.85 5.256, 7.443, 20.316, 24.017, 25.11, 12.800, 11.733, 9.662, 15.684, and 17.023±0.30 degrees 2θ angles.

Furthermore, DSC shows a characteristic endotherm at about 106 to 113° C. for Form C. The differential enthalpy analysis (DSC) of the ‘Form C’ was carried out using a TA Q100 instrument with a standard open pan arrangement, calibrated by reference to indium. For the calorimetric analysis, 2.0 mg of Form C was used, as obtained in EXAMPLE 2, in a crimped and pierced aluminum cup and scanned in a temperature range from 0 to 160 degree C. with a rate of heating of 5° C./minute.

The typical crystalline ‘Form C’ of Valsartan has X-ray powder diffraction pattern as substantially as shown in the FIG. 3 and the characteristic peaks with their 2θ values and corresponding d spacings and relative intensity in percentage are listed in Table 3 given below.

TABLE 3

| 2-theta values in degrees | D spacing | Percentage relative intensity |
|---|---|---|
| 5.256 | 16.8009 | 68.1 |
| 7.443 | 11.868 | 40.6 |
| 9.662 | 9.147 | 26.5 |
| 11.733 | 7.536 | 27.0 |
| 12.8001 | 6.9102 | 34.8 |
| 13.851 | 6.388 | 100 |
| 14.683 | 6.028 | 24.1 |
| 15.684 | 5.646 | 36.3 |
| 17.023 | 5.2044 | 33.9 |
| 20.316 | 4.367 | 39.9 |
| 24.017 | 3.702 | 27.6 |
| 25.111 | 3.543 | 28.2 |

In one embodiment, the present invention provides a process for preparing Valsartan in a stable crystalline form that denoted as ‘Form C’ having at least one characteristic listed in table 3. The process includes the steps of providing a suspension of Valsartan in an organic solvent selected from toluene, hexane, cyclohexane or the like, subjecting it to agitation at a temperature of about 0 degree to about 60 degrees for up to 110 hours duration till the required crystallinity is obtained and isolating the crystals formed in the suspension. Further, the process optionally includes the steps of reducing the temperature of the valsartan suspension and maintaining the suspension at reduced temperature, preferably less than room temperature for a holding time, preferably about 0-2 hours, most preferably about 1 hour.

In a preferred embodiment of the present invention, Valsartan is suspended in the solvent especially toluene at a temperature of about 20 to about 35° C. to form a fine suspension in said solvent, followed by maintaining the mass under constant stirring for about 24 hours to 115 hours. The amorphous or partially crystalline (partially crystalline is referred to a sample of Valsartan having amorphous Form as well as crystalline forms) may be used in the process to obtain the new ‘Form C’. Alternately, the sample of Valsartan can be subjected to shearing in the said solvents for a longer period of time. It has been found that when the shearing speed is increased, the conversion to crystalline ‘Form C’ is faster and normally with in a period of 24 hours, about 60% of amorphous valsartan converts to stable crystals of ‘Form C’. The suspension may be further cooled to a temperature of −5 to +5° C. to obtain pure ‘Form C’ of Valsartan. The ‘Form C’ crystals can then be separated from the mixture by conventional means such as filtration, centrifugation etc. and can be optionally dried at ambient or elevated temperatures.

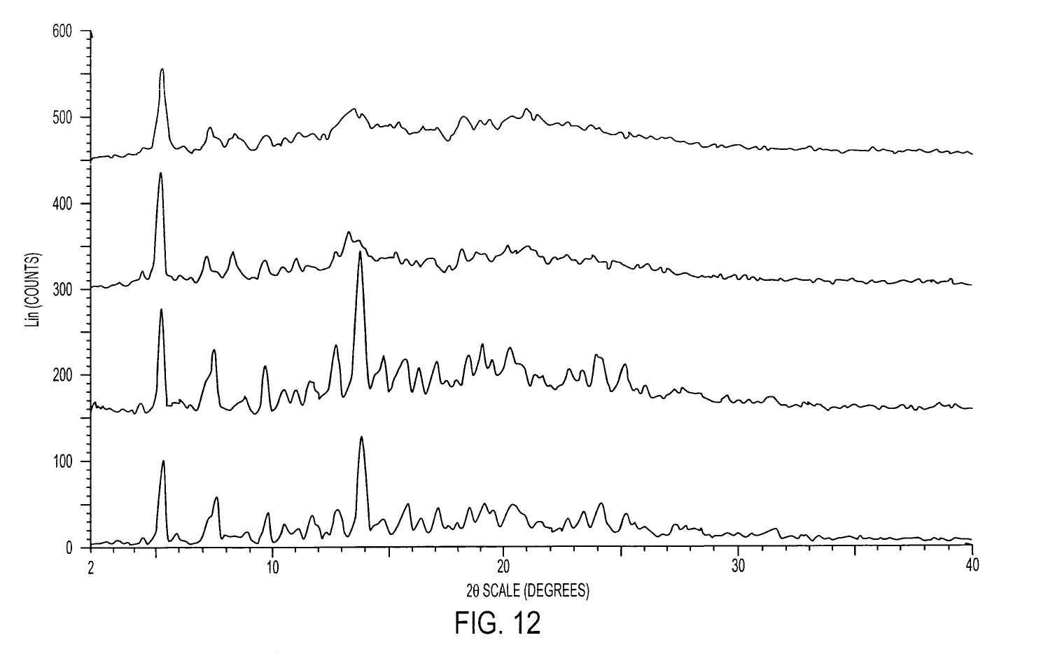

The conversion of the amorphous material was tracked at increased number of hours of maintenance of valsartan in toluene and the samples obtained at 24, 36, 48, 72, 96 hours were characterized using XRPD that are plotted in FIG. 12. One may observe the increase in the crystalline content in the Form C as the number of hours increases and the ‘Form C’ isolated are stable to processing further in a pharmaceutical product.

The hydrocarbon solvent (e.g. toluene) is used in about 5 to 30 volumes (mL) relative to the weight (g) of valsartan used and preferably the volume of solvent is 10 to 15 times that of valsartan. The starting valsartan is preferably stirred in the said solvent for about 50 to 115 hours, and more preferably about 72 to 100 hours. The ‘Form C’ crystals of valsartan is stable under experimental conditions.

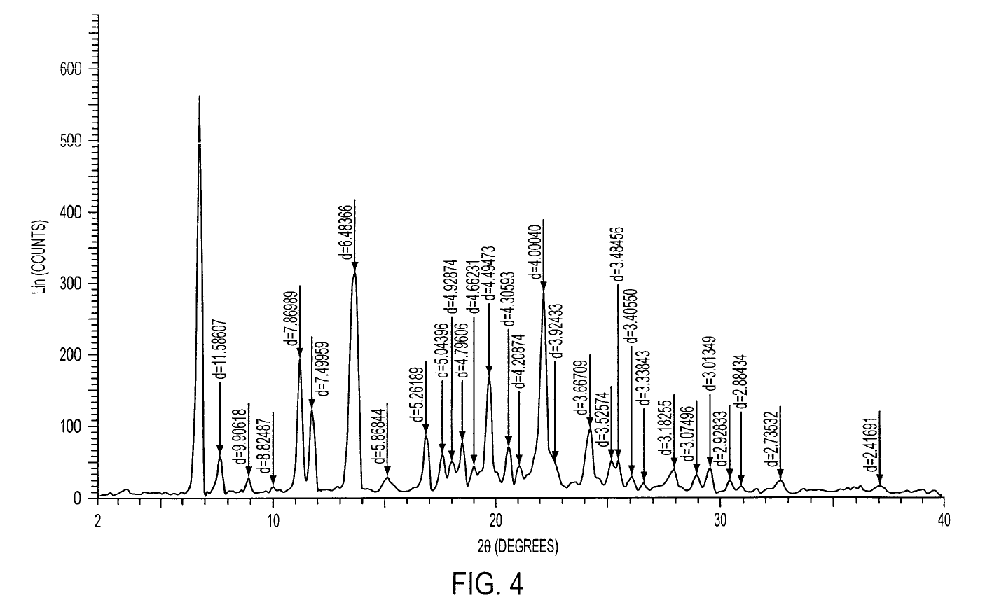

In yet another aspect, this invention provides one more novel Valsartan in a stable, high melting and distinguishable crystalline form that is denoted as “Valsartan ‘Form D’”. The character of this new form is identified and confirmed by PXRD patterns, FTIR and Differential Scanning Calorimeter thermogram (also referred as DSC) obtained from a sample thereof which are provided as FIGS. 4 & 10 respectively. The typical PXRD pattern shows at least one characteristic peak at about 6.50, 11.58, 16.63, 19.53, 21.99 and 24.04±0.2 degrees 2θ. More particularly the PXRD pattern shows characteristic peaks at 6.50 and 11.58±0.20 degrees 2θ. Furthermore, DSC shows a characteristic endotherm at about 129 to 135° C. for Form D. The differential enthalpy analysis (DSC) of the ‘Form D’ was carried out using a TA Q100 instrument with a standard open pan arrangement, calibrated by reference to indium. For the calorimetric analysis, 2.0 mg of Form D was used, as obtained in EXAMPLE 10, in a crimped and pierced aluminum cup and scanned in a temperature range from 0 to 160° C. with a rate of heating of 5° C./minute.

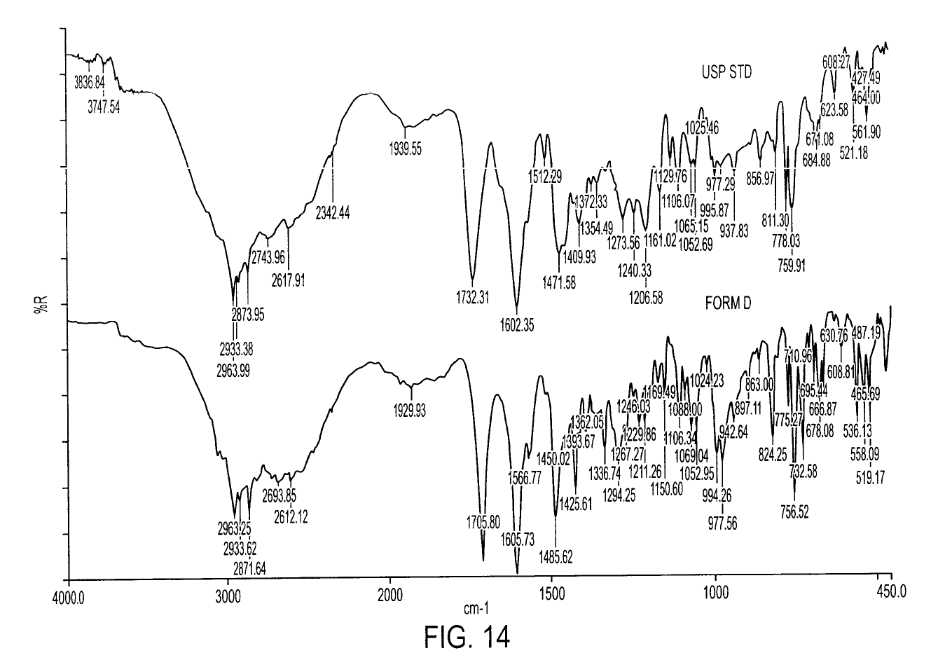

This more high melting crystalline form is also characterized and confirmed by FTIR spectra and the IR spectra were obtained as described above for the samples obtained from the process as in EXAMPLE 7. The FTIR spectra shows characteristic absorption at 1705, 1485, 1425, 1294, 824, 536, 678, and 666 cm⁻¹, which are absent in other forms. The IR spectra of USP reference sample and ‘Form D’ are recorded and compared in FIG. 14.

The crystalline ‘Form D’ of Valsartan typically has a X-ray powder diffraction pattern as substantially as shown in the FIG. 4 and the characteristic peaks with their 2θ values and corresponding d spacings and relative intensities in percentage are listed in the Table 4 below.

TABLE 4

| 2-theta values in degrees | d spacing | Percentage relative intensity |
|---|---|---|
| 6.509 | 13.56874 | 100 |
| 11.588 | 7.63035 | 26.3 |
| 16.633 | 5.32544 | 20.8 |
| 19.535 | 4.54053 | 34.1 |
| 21.997 | 4.03749 | 53.3 |
| 24.043 | 3.69837 | 18.5 |

An aspect of the present invention also provides a process for preparing high melting Valsartan in a stable crystalline form that denoted as ‘Form D’ having at least one characteristic listed above. The process includes the steps of providing a suspension of amorphous Valsartan or its mixture with other crystalline forms in an organic solvent selected from toluene, or its combination with hexane, cyclohexane, xylene, ethyl acetate, water or the like, subjecting it to agitation at a temperature starting from 0 degree to 60 degrees for longer than 114 hours duration until the required crystallinity is obtained and isolating the crystals formed in the suspension. Further, the process optionally includes the steps of reducing the temperature of the Valsartan suspension and maintaining the suspension at reduced temperature, preferably less than room temperature, for a holding time, preferably about 0-2 hours, more preferably about 1 hour.

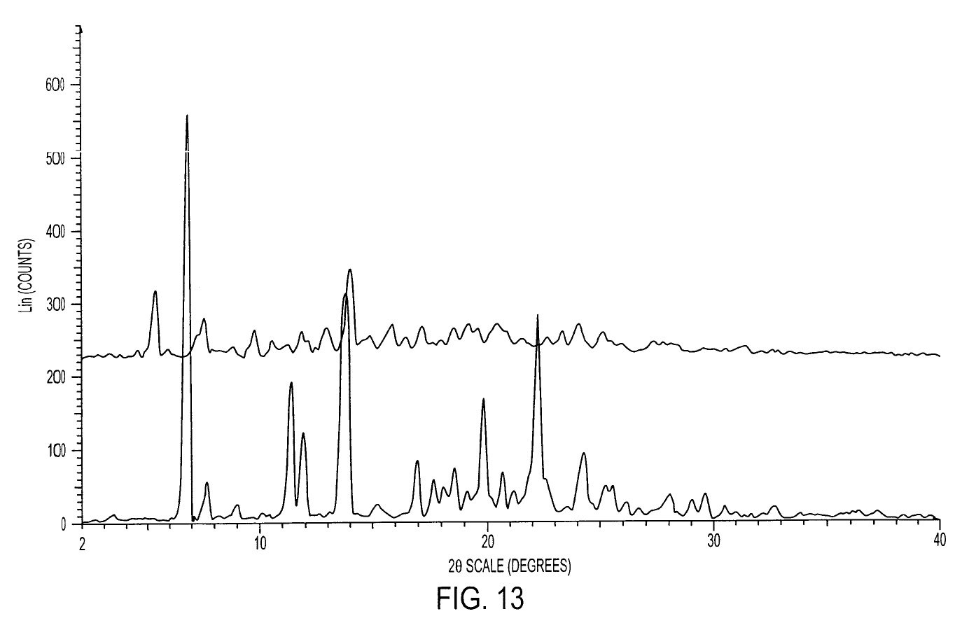

In a preferred embodiment of the present invention, valsartan is suspended in a solvent, preferably toluene, at a temperature of 20 to 35° C. to form a fine suspension in said solvent. The mass is then maintained under constant stirring for about 120 hours or more and may extend for several days. The agitation may be performed continuously or intermittently. When the sample crosses maintenance of 110 hours the ‘Form C’ crystals formed in the toluene completely transform into a higher melting polymorph of Valsartan. This intermediate polymorphic transition was identified and characterized using DSC and XRPD analyses (FIG. 13). This new crystalline Form D is found to be more crystalline, with a crystal content exceeding 90% and even has a crystal content of 95-98%.

Alternately, The new polymorphic form of Valsartan ‘Form D’ can be prepared by suspending amorphous or partially crystalline valsartan in toluene or its combination with xylene, hexane, ethyl acetate or water and seeding the mixture with seeds of ‘Form D’; agitating the mixture for a period of about 14 to 30 hours; and filtering out the crystalline ‘Form D’ Valsartan from the solvent or solvent mixture.

The amorphous or partially crystalline (partially crystalline is referred to a sample of Valsartan having amorphous Form as well as crystalline forms) may be used in the process to obtain the new ‘Form D’. The samples may be kept in toluene for longer period of time which may extend to several days and the ‘Form D’ valsartan was found stable under experimental conditions. It has been found that when the shearing speed is increased, the conversion of ‘Form C’ into ‘Form D’ takes place faster and normally with in a period of 5 hours after 110 hours, the stable crystals of ‘Form D’ is obtained. The ‘Form D’ crystals of valsartan can then be separated from the mixture by conventional means such as filtration, centrifugation etc. and can be optionally dried at ambient or elevated temperatures. This higher melting ‘Form D’ Valsartan is found to be denser and less soluble, and has a higher crystal content than any other forms.

In yet another aspect, the present invention relates to a process for preparing crystalline Valsartan denoted as ‘Form I.’ This method includes the steps of dissolving Valsartan in methyl propyl ketone to form a solution, cooling the solution/mixture to a temperature of about 25 to 35° C., and then further cooling to about 0 to 5° C. to obtain Valsartan in ‘Form I’. The Form I crystals can then be separated from the mixture by conventional means, such as filtration, and can be optionally dried at ambient or elevated temperatures. The Valsartan starting material can be dissolved in the solvent wherein heat is used to effect dissolution. Preferably the starting material is dissolved at about 50 to 55° C. to reflux temperature of the solvent.

The ‘Form I’ crystals of Valsartan typically has a PXRD showing characteristic peaks at 5.321, 12.98, 16.23, 19.421, 20.62, and 23.32 degrees 2θ and identical with the Form I disclosed in WO 04/083192.

The invention further provides a new process for obtaining pure amorphous form of Valsartan essentially having no endotherm in a Differential scanning calorimetry (DSC) thermogram. The new process of the invention comprises suspending valsartan in a solvent, toluene or xylene, at about 50° C., and further cooling the suspension to about 30° C. The glassy solid obtained can then be separated from the mixture by conventional means such as filtration, and dried at elevated temperatures, such as at 50° C., to obtain pure amorphous form of Valsartan.

The amorphous form of Valsartan is identified using PXRD that shows no significant/distinguishing peaks. It is further characterized by DSC and the thermogram of the amorphous form of Valsartan prepared according to the present invention shows no characteristic endotherms having an enthalpy of greater than 1 Joules. This amorphous form is denoted as pure amorphous Valsartan, which is different from the amorphous form obtained by the prior process at least in a DSC thermogram.

In a further aspect, the invention provides a compound obtainable by a process or method described above. Valsartan has been indicated for use in the following indications: hypertension, Cardiovascular diseases and Acute myocardial infarction. It may be used alone or concommitently with other classes of antihypertensive agents (ACE inhibitors or calcium channel blockers), such as amlodipine or its pharmaceutical salts, or diuretic agents, such as hydrochlorothiazide or its pharmaceutical salts, or antithormobolytics.

The invention thus provides substantially crystalline valsartan for use in treating hypertension, congestive heart failure and acute myocardial infarction. In the practice of the invention, the most suitable route of administration as well as the magnitude of a therapeutic dose of crystalline valsartan in any given case will depend on the nature and severity of the disease to be treated. The dose and dose frequency may also vary according to the age, body weight and response of the individual patient. In general a suitable oral dosage form may cover a dose range from 10 mg to 350 mg total daily dose, as administered in one single dose or equally divided doses. A preferred dosage range is from 50 mg to 250 mg. A higher dosage regimen may be used when the delivery of valsartan is intended to have a sustained release effect in patients.

Therefore in a further aspect, according to the present invention, Valsartan ‘Form A’ or ‘Form B’ or ‘Form C’ or ‘Form D’ or amorphous form and their solvates thereof are useful for treating patients with hypertension and for producing an anti-hypertensive/cardiovascular effect in mammals, including human patients. Valsartan ‘Form A’ or ‘Form B’ or ‘Form C’ or ‘Form D’ or amorphous form and their solvates thereof can be formulated into a variety of compositions for administration to humans and mammals.

Pharmaceutical compositions of the present invention contain Valsartan ‘Form A’ or ‘Form B’ or ‘Form C’ or ‘Form D’ or amorphous form and their solvates thereof and may optionally contain other crystalline forms and/or other active pharmaceutical ingredients, such as hydrochlorothiazide. In addition to the active ingredient(s), the pharmaceutical compositions of the present invention can contain one or more commonly used pharmaceutical excipients. Excipients are added to the composition for a variety of purposes well known in the art.

Valsartan ‘Form A’ or ‘Form B’ or ‘Form C’ or ‘Form D’ or amorphous form and their solvates thereof and their pharmaceutical composition can be administered for treatment of hypertension by any means that delivers the active pharmaceutical ingredient (s) to the site of the body where competitive inhibition of an AT-I receptor exerts a therapeutic effect on the patient. For example, administration can be oral, buccal, parenteral (including subcutaneous, intramuscular, and intravenous) rectal, inhalant and ophthalmic. Although the most suitable route in any given case will depend on the nature and severity of the condition being treated, the most preferred route of the present invention is oral. Valsartan ‘Form A’ or ‘Form B’ or ‘Form C’ or ‘Form D’ or amorphous form and their solvates thereof can be conveniently administered to a patient in oral unit dosage form and prepared by any of the methods well-known in the pharmaceutical arts.

Dosage forms include solid dosage forms, such as tablets, powders, capsules, sachets, troches and lozenges, as well as liquid syrups, suspensions and elixirs. The active ingredient (s) and excipients can be formulated into compositions and dosage forms according to methods known in the art.

Accordingly, valsartan ‘Form A’ or ‘Form B’ or ‘Form C’ or ‘Form D’ or amorphous form and their solvates thereof can be milled into a powder and be used in a pharmaceutical product/composition or physically modified such as by granulation to produce larger granules. Valsartan ‘Form A’ or ‘Form B’ or amorphous or ‘Form C’ or ‘Form D’ form and their solvates thereof can also be used to prepare a liquid pharmaceutical composition by dissolving or dispersing or suspending/emulsifying it in a pharmaceutically acceptable liquid medium such as water, glycerin, vegetable oil and the like as discussed in greater detail below.

When a dosage form such as a tablet is made by compaction of a powdered composition, the composition is subjected to pressure from a punch and dye. Solid and liquid compositions can also be dyed using any pharmaceutically acceptable colorant to improve their appearance and/or facilitate patient identification of the product and unit dosage level. In liquid pharmaceutical compositions of the present invention, Valsartan ‘Form A’ or ‘Form B’ or ‘Form C’ or ‘Form D’ or amorphous form and their solvates thereof and any other solid excipients are dissolved or suspended in a liquid carrier such as water, vegetable oil, alcohol, polyethylene glycol, propylene glycol or glycerin. Liquid pharmaceutical compositions can contain emulsifying agents to disperse uniformly throughout the composition an active ingredient or other excipient that is not soluble in the liquid carrier.

Selection of particular excipients and the amounts to use can be readily determined by the formulation scientist based upon experience and consideration of standard procedures and reference works in the field. The solid compositions of the present invention include powders, granulates, aggregates and compacted compositions.

Without further description, it is believed that one of ordinary skill in the art can, using the preceding description and the following illustrative examples, make and utilize the compounds of the present invention and practice the claimed methods. The following examples are given to illustrate the present invention. It should be understood that the invention is not to be limited to the specific conditions or details described in these examples.

### EXAMPLE 1

### Valsartan ‘Form A’

In a reaction vessel, 3 gm Valsartan (prepared as per method given in U.S. Pat. No. 5,399,578) was dissolved in 6 ml acetone at about 30° C. The mixture was stirred for about 30 minutes and 20 ml dichloromethane was added to the mixture. The mixture under agitation cooled to 0 to −5° C. A further 10 ml of MDC was added and maintained for about 3 to 4 hours. The white crystals obtained was filtered on a crucible and dried under vacuum at 30° C. The solid obtained shows a PXRD pattern of ‘Form A’ as in FIG. 1 and a DSC thermogram of FIG. 7.

### EXAMPLE 2

### Valsartan ‘Form B’

In a reaction vessel, 3 gm Valsartan (prepared as per method given in U.S. Pat. No. 5,399,578) was suspended in 30 ml toluene and was heated to reflux. Reflux was maintained for about 30 minutes and then the mixture under agitation was cooled to 25 to 30° C. The mixture was maintained at this temperature under agitation for about 30 to 34 hours. The white crystals obtained were filtered on a crucible after cooling to 0 to 5° C. and the crystals are dried under vacuum at 50° C. The solid obtained shows a PXRD pattern of ‘Form B’ as in FIG. 2 and a DSC thermogram of FIG. 8.

### EXAMPLE 3

### Valsartan ‘Form I’

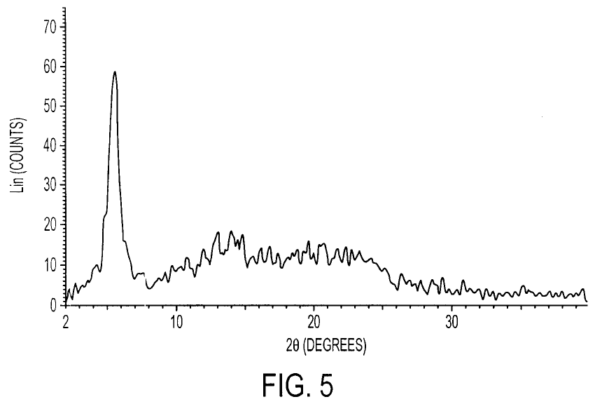

In a reaction vessel, 3 gm Valsartan (prepared as per method given in U.S. Pat. No. 5,399,578) was dissolved in 7 ml methyl propyl ketone and heated to 50 to 55° C. The mixture was stirred for about 30 minutes at 50 to 55° C. and then cooled to 0 to 5° C. The mixture was further maintained under cooling for about 3 hours. The solid obtained was filtered on a crucible and dried under vacuum at 30° C. The solid obtained shows a PXRD pattern of ‘Form I’ as in FIG. 5.

### EXAMPLE 4

### Amorphous Valsartan

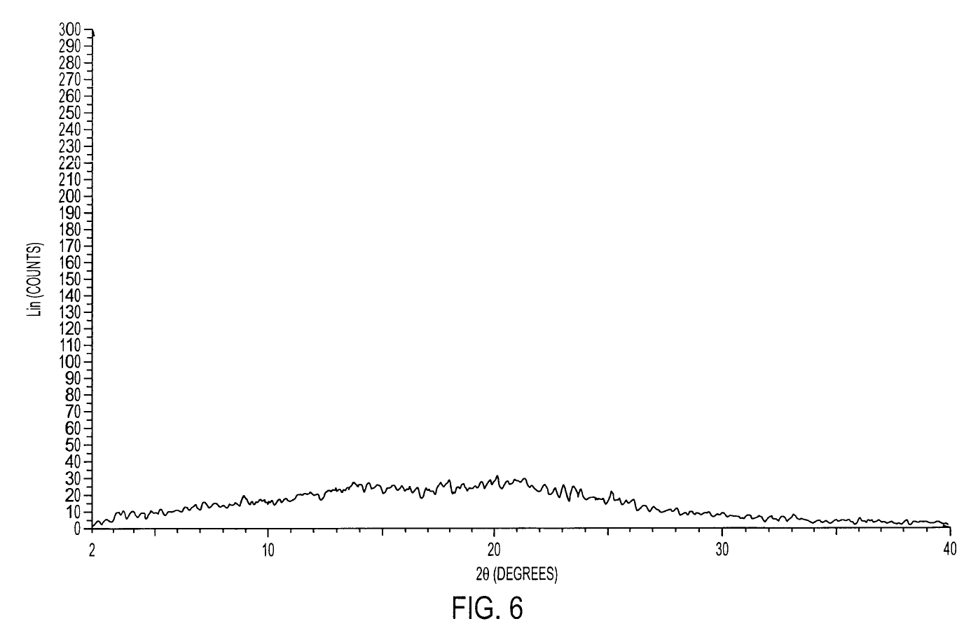

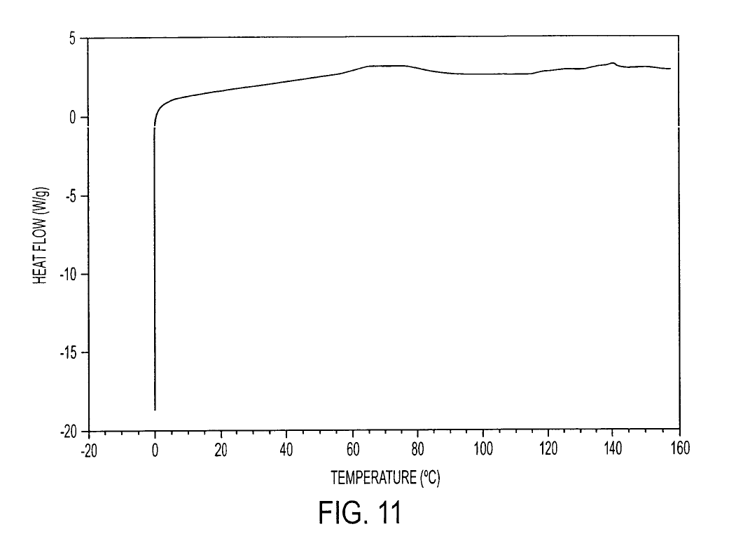

In a reaction vessel, 3 gm Valsartan (prepared as per method given in U.S. Pat. No. 5,399,578) was suspended in 30 ml toluene at about 40° C. The mixture was heated to 50° C. and stirred for about 30 minutes. The mixture under agitation was further cooled to 30° C. The white glass like solid obtained was filtered on a crucible and dried under vacuum at 50° C. The glass like amorphous solid obtained shows a PXRD pattern of ‘amorphous valsartan as in FIG. 6 and a DSC thermogram of FIG. 11.

### EXAMPLE 5

### Valsartan ‘Form C’

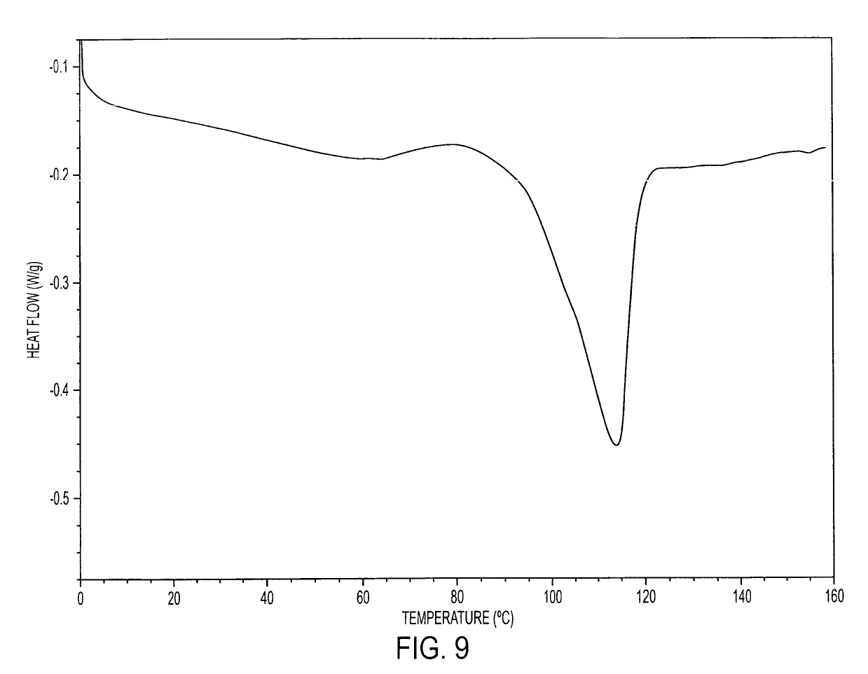

In a reaction vessel, 100 gm Valsartan (prepared as per method given in U.S. Pat. No. 5,399,578) was dissolved in 1200 ml toluene at about 45° C. The mixture was stirred for about 50 hours and cooled to a temperature of 0° C. The white crystals obtained was filtered on a crucible and dried under vacuum at 30° C. The solid obtained shows a PXRD pattern of ‘Form C’ as in FIG. 3 and a DSC thermogram of FIG. 9 Purity 99.8% and yield 98%.

### EXAMPLE 6

### Valsartan ‘Form C’

In a reaction vessel, 100 gm Valsartan (prepared as per method given in U.S. Pat. No. 5,399,578) was dissolved in 2000 ml toluene at about 30° C. The mixture was stirred for about 100 hours the white crystals obtained was filtered on a crucible and dried under vacuum at 30° C. The solid obtained shows a PXRD pattern of ‘Form C’ as in FIG. 3 and a DSC thermogram of FIG. 9 Purity 99.8% and yield 99%.

### EXAMPLE 7

### Valsartan ‘Form C’

In a reaction vessel, 100 gm Valsartan (prepared as per method given in U.S. Pat. No. 5,399,578) was dissolved in 1000 ml toluene at about 30° C. The mixture was stirred for about 72 hours the white crystals obtained was filtered on a crucible and dried under vacuum at 30° C. The solid obtained shows a PXRD pattern of ‘Form C’ as in FIG. 3 and a DSC thermogram of FIG. 9 Purity 99.8% and yield 99%.

### EXAMPLE 8

### Valsartan ‘Form D’

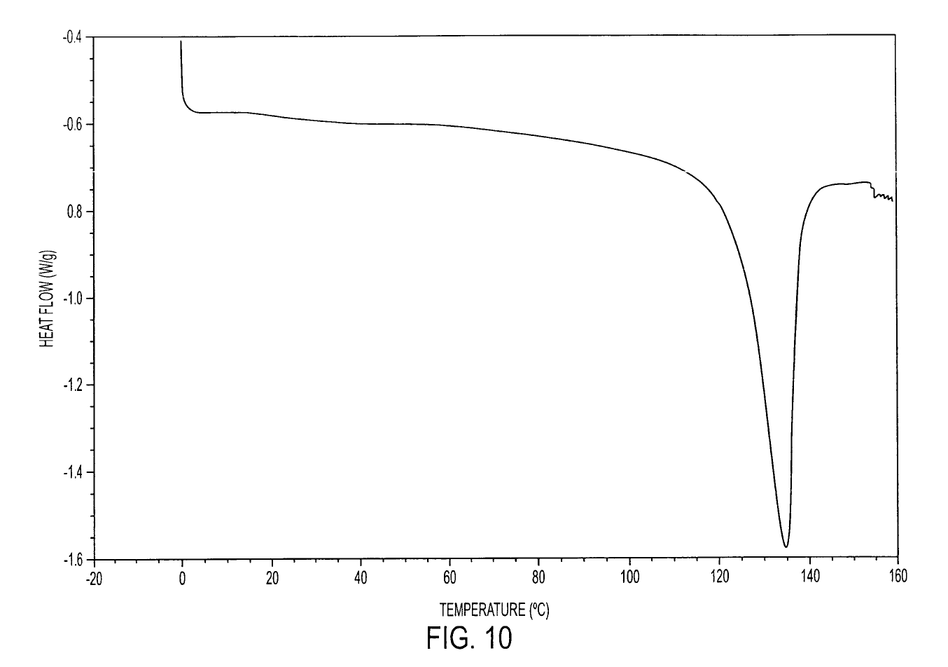

In a reaction vessel, 25 gm Valsartan (prepared as per method given in U.S. Pat. No. 5,399,578) was dissolved in 125 ml toluene at about 30° C. The mixture was stirred for about 120 hours the white crystals obtained was filtered on a crucible and dried under vacuum at 30° C. The solid obtained shows a PXRD pattern of ‘Form D’ as in FIG. 4 and a DSC thermogram of FIG. 10. Purity 99.8% and yield 99%.

### EXAMPLE 9

### Valsartan ‘Form D’

In a reaction vessel, 25 gm Valsartan (prepared as per method given in U.S. Pat. No. 5,399,578) was dissolved in 250 ml toluene at about 30° C. Seed the reaction mass with Form D obtained as per example 7 and the mixture was stirred for about 24 hours the white crystals obtained was filtered on a crucible and dried under vacuum at 30° C. The solid obtained shows a PXRD pattern of ‘Form D’ as in FIG. 4 and a DSC thermogram of FIG. 10. Purity 99.8% and yield 99%.

### EXAMPLE 10

### Valsartan ‘Form D’

In a reaction vessel, 25 gm Valsartan (prepared as per method given in U.S. Pat. No. 5,399,578) was dissolved in mixture of 50 ml toluene and 50 ml ethyl acetate at about 30° C. Seed the reaction mass with Form D obtained as per example 7 and the mixture was stirred for about 24 hours the white crystals obtained was filtered on a crucible and dried under vacuum at 30° C. The solid obtained shows a PXRD pattern of ‘Form D’ as in FIG. 4 and a DSC thermogram of FIG. 10. Purity 99.8% and yield 99%.

### EXAMPLE 11

### Valsartan ‘Form D’

In a reaction vessel, 25 gm Valsartan (prepared as per method given in U.S. Pat. No. 5,399,578) was dissolved in mixture of 75 ml toluene and 75 ml water at about 30° C. Seed the reaction mass with Form D obtained as per example 7 and the mixture was stirred for about 24 hours the white crystals obtained was filtered on a crucible and dried under vacuum at 30° C. The solid obtained shows a PXRD pattern of ‘Form D’ as in FIG. 4 and a DSC thermogram of FIG. 10. Purity 99.8% and yield 99%.

### EXAMPLE 12

### Characterization of Valsartan ‘Form D’

The stable crystalline form of valsartan designated as ‘Form D’ is selected for exemplifying the beneficial effects of the new forms as compared to the amorphous form as illustrated by the following tests.

Characterization for the Degree of Crystallinity for Valsartan Samples of ‘Form D’ Crystals.

Characterization for the degree of crystallinity of Valsartan crystalline ‘Form D’ was performed by using conventional Bragg Brentano XRPD. The XRPD evaluation technique is used to provide the best estimates for the degree of crystallinity by classical evaluation of the peak area with the minimum detection level of <1% w/w according to the methods reported in Fix, K. J. Steffens, Drug Development & Industrial Pharmacy, 30, 5, 2004, 513-523 and J. M.-Bernardo, S. G.-Granda, M. B.-Jasanada, I. Llorente, L. Llavona, ARKIVOC 2005 (ix) 321-331.

The x-ray powder diffraction pattern for the physically prepared binary mixtures (in the interval of 90-100%) was obtained. At least three replicate measurements were performed for each sample. The percentage crystallinity of samples obtained according to the present invention is calculated to be as greater as 96-99% or more.

Comparative Bulk Density Determination.

The bulk density of sample of crystalline ‘Form D’ valsartan was compared with amorphous form of valsartan and the results are summarized in Table 5 below:

TABLE 5

| | Bulk density | Tapped density |
|---|---|---|
| Sample | (gm/ml) | (1250 taps) gm/ml |
| ‘Form D’ | 0.4561 | 0.6139 |
| Vlasartan | | |
| Amorphous | 0.37 | 0.504 |
| valsartan | | |

The Equilibrium Solubility Determination.

The equilibrium solubility for crystalline ‘Form D’ valsartan was tested along side the standard amorphous valsartan in solvent 1-octanol by the following procedure. Saturated solutions were prepared by performing dissolution of excess amounts of each polymorph into 5 ml of 1-octanol solvent in a vial. The sample solutions were placed on a thermostatic water-bath maintained at 23±0.5° C. for 15, 30, 45, 60 minutes under magnetic stirring conditions. Aliquots of solutions were withdrawn with a syringe, filtered through microfilter membrane and appropriately diluted with 1-octanol. The concentration of the drug in 1-octanol was measured on a UV spectrophotometer and data was compiled.

The saturation/equilibrium solubility was confirmed by preparing saturated solutions in glass vials by adding excess of each form into an appropriate volume of solvent so that the sediment was left after vigorous shaking for 1.5 hours on a thermostatic magnetic stirrer. The samples were centrifuged and filtered through microfilter membrane filter, diluted with the 1-octanol and then quantitatively determined by UV absorption.

The saturation solubility data of Crystalline ‘Form D’ and amorphous valsartan are given in Table 6 below:

TABLE 6

| | solubility |
|---|---|
| Sample | (mg/ml) |
| ‘Form D’ | 103 mg |
| Vlasartan | |
| Amorphous | 152 mg |
| valsartan | |

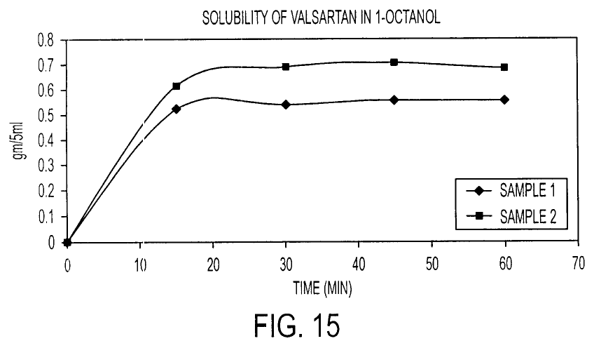

Further the time dependent equilibrium solubility of crystalline ‘Form D’ and the standard amorphous valsartan are given in FIG. 15 distinguishing the solubility pattern of ‘Form D’ valsartan from amorphous valsartan.

Intrinsic Dissolution Studies

Dissolution studies were carried by stationary disk method, as disclosed in Chem. Pharm Bull. 30(7), 1982, 2618-2620, which is incorporated herein by reference, using a rotating paddle dissolution apparatus (basket). 50 mg of the powder samples of crystalline ‘Form D’ as well as standard amorphous valsartan were filled into empty hard gelatin capsules (constant weight) and tested for intrinsic dissolution in three media of pH 1.2, 4.5 and 6.8 buffer solutions. As the valsartan is practically insoluble in water, buffer solutions of pH 1.2, 4.5, and 6.8 were prepared using a mixture of methanol and a phosphate buffer (30:70 ratio). The samples of each crystalline ‘Form D’ and amorphous form of valsartan were tested under the following conditions: Dissolution medium (500 ml) at 37° C. and 100 rpm stirrer speed. Identical conditions were maintained for each polymorph studied. 10 ml aliquots from each sample were removed at time intervals of 10, 20, 30, 40, 50, and 60 minutes and filtered. These aliquots were then analyzed using HPLC analysis method of valsartan as per US pharmacopoeia by diluting aliquots and standards appropriately to obtain solution of about 10 ppm concentration.

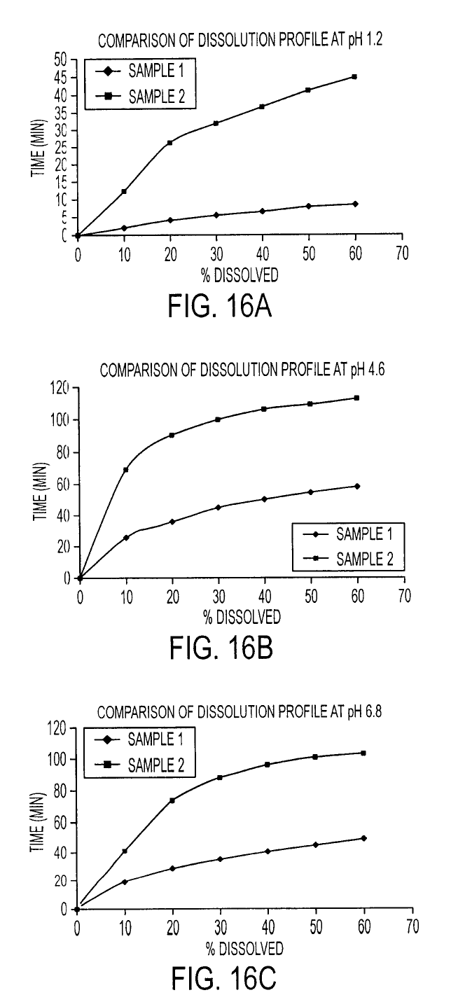

The dissolution profile of crystalline ‘Form D’ and amorphous valsartan in 1.2, 4.5 and 6.8 pH buffers are given in FIG. 16.

The above studies indicates that the crystalline Form D of valsartan is at least 4.5 fold less soluble than the amorphous valsartan at pH 1.2 and at least 2 fold less soluble under pH conditions of 4.5 and 6.8.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

We claim:

1. A crystalline Form A of valsartan having a powder X-Ray diffraction pattern (PXRD) containing peaks at about 6.7488, 14.237, 20.87, 21.807 and 22.256 degrees 2θ.

2. A crystalline Form A of Valsartan as claimed in claim 1, further having a thermal analysis results in a Differential Scanning Calorimeter (DSC) thermogram taken at a heating rate of 5 degree Celsius per minute in a open pan that exhibits a melting endotherm at 95 to 96° C.

3. A method for making crystalline Form B of valsartan comprising the steps of:
i) preparing a solution of amorphous or crystalline valsartan in a first solvent selected from the group consisting of acetone, methyl propyl ketone, and their mixture thereof;
ii) mixing with dichloromethane till a suspension is resulted; and
iii) separating said crystalline form of valsartan Form A from the solvents.

4. A crystalline Form B of valsartan having a powder X-Ray diffraction pattern (PXRD) containing peaks at about 5.810, 9.815, 11.463, 13.937 and 17.420 degrees 2θ.

5. A crystalline Form B of Valsartan as claimed in claim 4, further having a thermal analysis results in a Differential Scanning calorimeter thermogram taken at a heating rate of 5 degree Celsius per minute in a open pan that exhibits a melting endotherm at about 103° C.

6. A method for making crystalline Form B of valsartan as comprising the steps of:
i) providing an emulsion or suspension of Valsartan in an organic solvent at a first temperature above 85° C.;
ii) reducing the temperature of the emulsion or suspension to a second temperature below 40° C.;
iii) maintaining the mixture at the second temperature for about 24 to 40 hours;
iv) further reducing the temperature of the stirred suspension to a third temperature range below 20° C.; and
v) isolating crystalline Form B of Valsartan by filtration.

7. A crystalline Form C of valsartan having a powder X-Ray diffraction pattern (PXRD) containing peaks at about 13.85, 5.256, 7.443, 20.316, 24.017, 25.11, 12.800, 11.733, 9.662, 15.684, and 17.023 degrees 2θ.

8. A crystalline Form C of valsartan as claimed in claim 7, further having a thermal analysis results in a Differential Scanning calorimeter thermogram taken at a heating rate of 5 degree Celsius per minute in a open pan that exhibits a melting endotherm at about 106-113° C. temperature.

9. The crystalline Form C of valsartan as claimed in claim 7, wherein the said crystalline form contains at least 50% crystals of Form C.

10. A method for making crystalline Form C of valsartan comprising the steps of:
i) providing a suspension or emulsion of valsartan in a hydrocarbon solvent;
ii) agitating the suspension for a period of 24 hours to 110 hours; and
iii) separating said new crystalline ‘Form C’ valsartan.

11. A crystalline Form D of valsartan having a powder X-Ray diffraction pattern (PXRD) containing peaks at about 6.50, 11.58, 16.63, 19.53, 21.99 and 24.04 degrees 2θ.

12. A crystalline Form D of valsartan having a thermal analysis results in a Differential Scanning calorimeter thermogram taken at a heating rate of 5 degree Celsius per minute in a open pan that exhibits a melting endotherm at about 129-135° C.

13. A crystalline Form D of valsartan having absorptions at 1705, 1485, 1425, 1294, 824, 536, 678, and 666 cm⁻¹ on a Fourier Transform (FT) Infra-Red spectra recorded between 4000 cm⁻¹ to 400 cm⁻¹.

14. The crystalline Form D of valsartan as claimed in claim 11, wherein the crystal content exceeds 85%.

15. A crystalline valsartan having a crystal content exceeding 90%.

16. A method for making crystalline Form D of valsartan comprising the steps of:
i) providing valsartan in an organic solvent;
ii) agitating the mixture, optionally with seed crystals of Form D; and
vi) separating said new crystalline ‘Form D’ valsartan.

17. The method as claimed in claim 16, wherein the solvent is hydrocarbon such as toluene or its mixture with hexane, xylene, ethyl acetate, or water.

18. The method as claimed in claim 16, wherein the mixture is agitated for over 115 hours.

19. A pharmaceutical composition or dosage form comprising the crystalline Form C and/or Form D of valsartan, and optionally a second active pharmaceutical drug.

20. The pharmaceutical composition or dosage form as claimed in claim 19, wherein the second active pharmaceutical drug is hydrochlorothiazide, amlodipine or its pharmaceutically acceptable salts.
